(19) United States
(12) Patent Application Publication
(10) Pub. No.: US 2008/0177203 A1
(43) Pub. Date: Jul. 24, 2008

(54) SURGICAL NAVIGATION PLANNING SYSTEM AND METHOD FOR PLACEMENT OF PERCUTANEOUS INSTRUMENTATION AND IMPLANTS

(75) Inventor: Ronald A. von Jako, Saugus, MA (US)

Correspondence Address: PETER VOGEL;GE HEALTHCARE, 3000 N. GRANDVIEW BLVD., SN-477, WAUKESHA, WI, 53188, US

(73) Assignee: GENERAL ELECTRIC COMPANY, Schenectady, NY (US)

(21) Appl. No.: 11/617,861

(22) Filed: Dec. 29, 2006

Related U.S. Application Data
(63) Continuation-in-part of application No. 11/615,440, filed on Dec. 22, 2006.

(51) Int. Cl. A61B 17/58

(52) U.S. Cl. 600/587

(57) ABSTRACT

A system and method for placement of at least one implant comprising an imaging system configured for taking at least one image of a patient; a navigation system configured for tracking position and orientation of at least one implant; a computer configured to measure and calculate the position and orientation of the at least one implant; and a display configured to display the at least one image of the patient and superimpose a graphical representation of the at least one implant with position and orientation information of the at least one implant on the at least one image of the patient.

### CROSS-REFERENCE TO RELATED APPLICATIONS

This application is a continuation-in-part of and claims priority to U.S. patent application Ser. No. 11/615,440, filed Dec. 22, 2006, the disclosure of which is incorporated herein by reference.

### BACKGROUND OF THE INVENTION

This disclosure relates generally to image-guided surgery (or surgical navigation). In particular, this disclosure relates to a surgical navigation planning system and method for placement of percutaneous instrumentation and implants.

Medical navigation systems track the precise location of surgical instruments in relation to multidimensional images of a patient's anatomy. Additionally, medical navigation systems use visualization tools to provide the surgeon with co-registered views of these surgical instruments with the patient's anatomy. The multidimensional images of a patient's anatomy may include computed tomography (CT) imaging data, magnetic resonance (MR) imaging data, positron emission tomography (PET) imaging data, ultrasound (UL) imaging data, X-ray imaging data, or any other suitable imaging data, as well as any combinations thereof.

Medical navigation technology has been applied to various areas of the body including the spinal column. Surgical procedures involving the spinal column may be used, for example, to stabilize and/or fuse portions of the spine or to correct various spinal deformities or degenerative conditions. Spinal surgeons and the spinal industry have developed minimally invasive surgical (MIS) techniques and technologies, such as posterior fixation delivery devices that enable percutaneous placement of pedicle screws and rods for stabilizing the spine.

The modern trend of treating chronic degenerative spinal disease by pedicle screws and rods encompasses small incisions in the back without the need of larger incisions and significant muscle disruption to accomplish spinal fixation and fusion. New instruments have been developed by the spinal industry to facilitate placement through least invasive access approaches that has lead to removal of the direct visualization of the bony anatomy by the surgeon. This has increased the reliance on X-ray fluoroscopy and made an already technical demanding procedure more complex. The need to dorsally target the spinal entry points for pedicle implant screws depends on good X-ray visualization and real-time planning. Surgical navigation helps to accomplish this through near real-time planning on saved X-ray images by virtual instruments superimposed over previously acquired X-ray images. Once the pedicle screw implants are all in position, a rod system is deployed subcutaneously to engage the anchored screws in a linear direction between adjacent spinal segments and rarely in a horizontal direction or from one side of the vertebrae to the opposite vertebral side. This helps reach the surgical goal of realigning the spinal vertebral segment(s) to its natural curvature and geometry, while at the same time fixing it into a frozen position with screws and rods to allow spinal fusion to occur between one more spinal levels. The current methods of targeting the pedicle screw implant with different size rods, are technically challenging and sometimes time consuming for the surgeon and can lead to poor alignment and potentially a weakened construct, that may eventually effect both the spinal fusion success and or failure of the construct there after.

It is difficult for a surgeon or other medical practitioners to see medical instruments or implanted devices during percutaneous procedures. For spinal fusion, interconnecting rods are inserted into implanted pedicle screws. These rods need to be pre-selected or cut to a specific size. Making approximate measurements for anatomical differences and rod length with or without jigs (rod guide sleeves) are not fully reliable and direct access to the implanted screws can be problematic in percutaneous MIS procedures and ultimately for desired compression and/or distraction and are therefore also prone to trial-and-error methods.

Despite advances in preoperative planning software and surgical instrument systems, many measurements are still made during a surgical procedure. For example, a surgeon may decide what diameters and lengths of pedicle screws he or she will use for a spinal fusion based on anatomic measurements off of a pre-acquired image of a patient's spine. In addition, compressions and other conditions affect measurements of interconnecting rods that lock adjacent vertebrae together, so it is difficult to measure the correct length and curvature of these rods accurately prior to surgery. Therefore, the length and curvature of the interconnecting rods are determined either by intraoperative trial and error fitting, or by the use of a surgical compass device or other instrument to make direct measurements. These techniques for placement of pedicle screws and fitting of interconnecting rods potentially contribute to extended time of the procedure and higher risk of infection.

Therefore, there is a need for a system and method for a surgical navigation planning system and method for placement of percutaneous instrumentation and implants.

### BRIEF DESCRIPTION OF THE INVENTION

In an embodiment, a method for placement of an implant comprising measuring a position of at least two implants; calculating a distance between the position of the at least two implants; and displaying the distance to a user.

In an embodiment, a system for placement of at least one implant, said system comprising an imaging system configured for taking at least one image of a patient; a navigation system configured for tracking position and orientation of at least one implant; a computer configured to measure and calculate the position and orientation of the at least one implant; and a display configured to display the at least one image of the patient and superimpose a graphical representation of the at least one implant with position and orientation information of the at least one implant on the at least one image of the patient.

In an embodiment, a computer-readable medium having a set of instructions for execution on a computer, said set of instructions comprising a tracking routine for measuring the position and orientation of at least one implant; a measurement routine for measuring differences between the position and orientation of the at least one implant; a calculation routine for calculating curves and trajectories for implant placement; and a display routine for displaying an X-ray and or combination of other image modalities (MR, PET, US) image (2D or 3D) of a patient and superimposing on the image a graphical representation (such as a template and/or 3D CAD model) of the at least one implant with position and orientation information, and curve and trajectory information.

In an embodiment, a method for implant placement and measurement comprising measuring the positions (x, y, z) of at least two implants; and calculating a distance between the two positions of the at least two implants.

In an embodiment, a method for implant placement and measurement comprising measuring the positions (x, y, z) of three or more implants; and calculating a best fit curve between the three or more positions of the three or more implants.

In an embodiment, a method for implant placement and measurement comprising measuring the positions (x, y, z) of at least two implants; measuring the orientations (roll, pitch, yaw) of the at least two implants; and calculating a best fit curve between the at least two positions of the at least two implants and the at least two orientations of the at least two implants.

The method further comprising measuring the pedicle screw head surface's cortex height between the two pedicle distances. The method further comprising measuring the positional distance and angles of each instrument guide sleeve to the pedicle screw heads.

Tracking and measuring orientation between jigs such as guide sleeves attached to implanted pedicle screws and for ensuring positive engagement between guide sleeves and pedicle screws to measure any deflections back and forth between parallel or adjacent guide sleeves to assist in proper seating of the pedicle screw rod ends into each screw head minimizing the need for x-ray fluoroscopy control shots to confirm a properly seated rod and screw construct. Advanced forms may obviate the need for guide sleeves.

Various other features, objects, and advantages of the invention will be made apparent to those skilled in the art from the accompanying drawings and detailed description thereof.

### BRIEF DESCRIPTION OF THE DRAWINGS

FIG. 1 is an exemplary schematic diagram of an embodiment of an imaging and navigation system;

FIG. 2 is an exemplary block diagram of an embodiment of an imaging and navigation system;

FIG. 3 is an exemplary display of an example of a user interface, such as a display, displaying an image with implant position and orientation information superimposed on the image;

FIG. 4A is an exemplary diagram of an embodiment of a pedicle screw interconnecting rod holder holding a pedicle screw interconnecting rod;

FIG. 4B is an exemplary diagram illustrating an embodiment of the placement of a pedicle screw interconnecting rod within at least two pedicle screw heads;

FIG. 5A is an exemplary diagram illustrating an embodiment of a portion of a spine with pedicle screws and a pedicle screw interconnecting rod placed within the pedicle screw heads;

FIG. 5B is an exemplary diagram illustrating an embodiment of a portion of a spine with pedicle screws and a pedicle screw interconnecting rod placed within the pedicle screw heads; and

FIG. 6 is an exemplary flowchart of an embodiment of a method for measuring and calculating implant position and orientation information.

### DETAILED DESCRIPTION OF THE INVENTION

In minimally invasive surgical (MIS) procedures, access to the body is obtained through one or more natural openings or small percutaneous incisions. Surgical instruments or implants are inserted through these openings and directed to a region of interest within the body. Direction of the surgical instruments or implants through the body is facilitated by navigation technology wherein the real-time location of a surgical instrument or implant is measured and virtually superimposed on an image of the region of interest. The image may be a pre-acquired image, or an image obtained in near real-time or real-time using known imaging technologies such as computed tomography (CT), magnetic resonance (MR), positron emission tomography (PET), ultrasound (US), X-ray, or any other suitable imaging technology, as well as any combinations thereof.

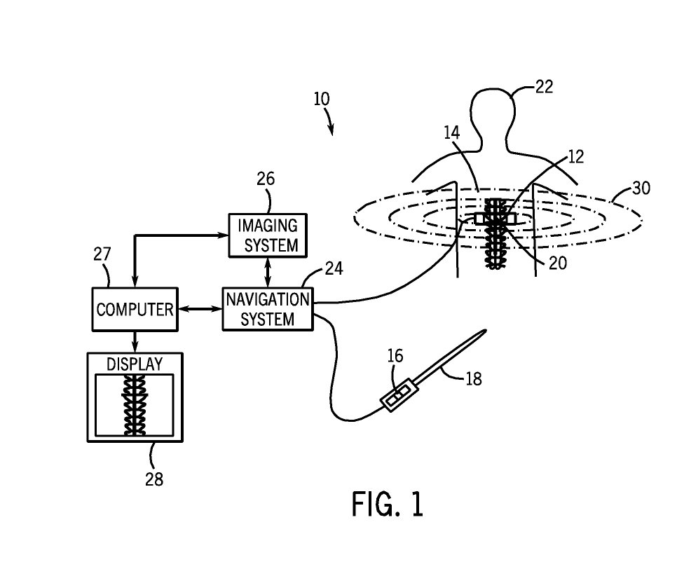

Referring now to the drawings, FIG. 1 is an exemplary schematic diagram of an embodiment of an imaging and navigation system. The system 10 includes at least one electromagnetic field generator 12 positioned proximate to a surgical field of interest 14, at least one electromagnetic sensor 16 attached to at least one navigated surgical instrument 18 to which a pedicle screw or other implant may be attached, the at least one electromagnetic sensor 16 communicating with and receiving data from the at least one electromagnetic field generator 12, a navigation system 24 coupled to and receiving data from the at least one electromagnetic sensor 16 and the at least one electromagnetic field generator 12, an imaging system 26 coupled to the navigation system 24 for performing imaging on a patient 22 in the surgical field of interest 14, a computer 27 coupled to the navigation system 24 and the imaging system 26, and a display 28 coupled to the computer 27 for displaying imaging and tracking data from the imaging system 26 and the navigation system 24.

The at least one electromagnetic field generator 12 may be attached to a registration apparatus 20 that may be attached to the patient 22 in the surgical field of interest 14. The at least one electromagnetic field generator 12 creating a local reference frame for the navigation system 24 around the patient's anatomy.

The display 28 may be configured to show the real-time position and orientation of a model of the at least one surgical instrument 18 or at least one implant attached to the tip or end of the at least one surgical instrument 18 on a registered image of the patient's anatomy. The model of the at least one surgical instrument 18 or at least one implant may appear as a line rendering, a few simply shaded geometric primitives (e.g., a parametric model containing two cylinders representing the screw head and body of a pedicle screw), or a realistic 3D model from a computer-aided design (CAD) file.

In an exemplary embodiment, the imaging system 26 and the navigation system 24 may be integrated into a single integrated imaging and navigation system with integrated instrumentation and software.

The system 10 enables a surgeon to continually track the position and orientation of the surgical instrument 18 or an implant attached to the surgical instrument 18 during surgery. An electromagnetic field 30 is generated around the at least one electromagnetic field generator 12. The at least one electromagnetic sensor 16 detects the electromagnetic field 30 generated by the at least one electromagnetic field generator 12 attached to the registration apparatus 20. The at least one electromagnetic sensor 16 may be an electromagnetic field receiver. The electromagnetic field receiver may be a receiver array including at least one coil or at least one coil pair and electronics for digitizing magnetic field measurements detected by the receiver array. The at least one electromagnetic field generator 12 may be an electromagnetic field transmitter. The electromagnetic field transmitter may be a transmitter array including at least one coil or at least one coil pair. It should, however, be appreciated that according to alternate embodiments the registration apparatus 20 may include at least one electromagnetic field receiver attached thereto and the surgical instrument 18 may include at least one electromagnetic field transmitter attached thereto.

The magnetic field measurements can be used to calculate the position and orientation of the surgical instrument 18 or an implant according to any suitable method or system. After the magnetic field measurements are digitized using electronics, the digitized signals are transmitted from the at least one electromagnetic sensor 16 to the navigation system 24. The digitized signals may be transmitted from the at least one electromagnetic sensor 16 to the navigation system 24 using wired or wireless communication protocols and interfaces. The digitized signals received by the navigation system 24 represent magnetic field information detected by the at least one electromagnetic sensor 16. The digitized signals are used to calculate position and orientation information of the surgical instrument 18 or implant. The position and orientation information is used to register the location of the surgical instrument 18 or implant to acquired imaging data from the imaging system 26. The position and orientation data is visualized on the display 28, showing in real-time the location of the surgical instrument 18 or implant on pre-acquired or real-time images from the imaging system 26. The acquired imaging data from the imaging system 26 may include CT imaging data, MR imaging data, PET imaging data, ultrasound imaging data, X-ray imaging data, or any other suitable imaging data, as well as any combinations thereof. In addition to the acquired imaging data from various modalities, real-time imaging data from various real-time imaging modalities may also be available.

The navigation system 24 is illustrated conceptually and may be implemented using any combination of dedicated hardware boards, digital signal processors, field programmable gate arrays, and processors. Alternatively, the navigation system 24 may be implemented using an off-the-shelf computer with a single processor or multiple processors, with the functional operations distributed between processors. As an example, it may be desirable to have a dedicated processor for position and orientation calculations as well as a processor for visualization operations. The navigation system 24 may be an electromagnetic navigation system utilizing electromagnetic navigation technology. However, other tracking or navigation technologies may be used.

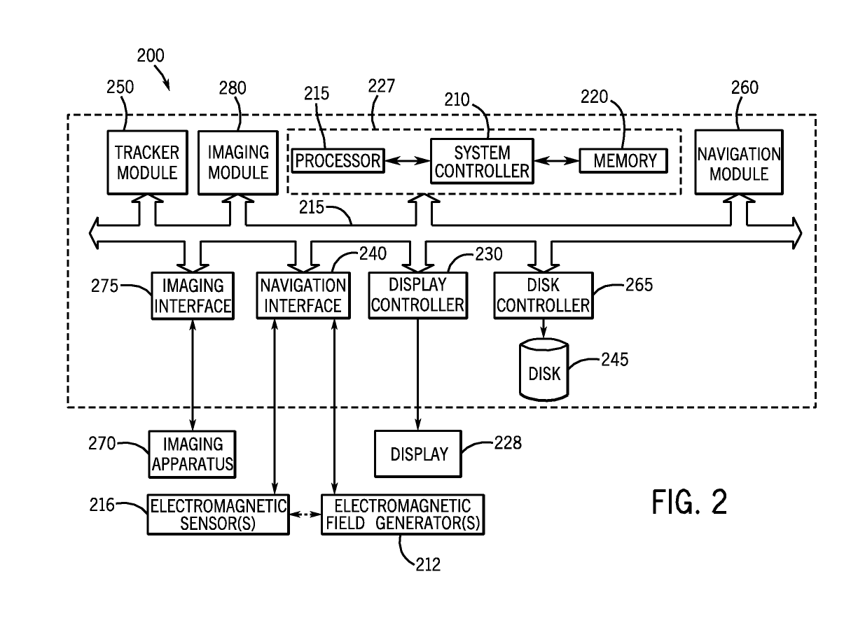

FIG. 2 is an exemplary block diagram of an embodiment of an imaging and navigation system 200. The imaging and navigation system 200 is illustrated conceptually as a collection of modules, but may be implemented using any combination of dedicated hardware boards, digital signal processors, field programmable gate arrays, and processors. Alternatively, the modules may be implemented using an off-the-shelf computer with a single processor or multiple processors, with the functional operations distributed between the processors. As an example, it may be desirable to have a dedicated processor for position and orientation calculations as well as a dedicated processor for visualization operations. As a further option, the modules may be implemented using a hybrid configuration in which certain modular functions are performed using dedicated hardware, while the remaining modular functions are performed using an off-the-shelf computer. In the embodiment shown in FIG. 2, the system includes a single computer 227 having a processor 215, a system controller 210 and memory 220. The operations of the modules may be controlled by the system controller 210.

The imaging and navigation system 200 includes at least one electromagnetic field generator 212 that is coupled to a navigation interface 240. The at least one electromagnetic field generator 212 generates at least one electromagnetic field that is detected by at least one electromagnetic field sensor 216. The navigation interface 240 receives digitized signals from at least one electromagnetic sensor 216. The navigation interface 240 includes at least one Ethernet port. The at least one Ethernet port may be provided, for example, with an Ethernet network interface card or adapter. However, according to various alternate embodiments, the digitized signals may be transmitted from the at least one electromagnetic sensor 216 to the navigation interface 240 using alternative wired or wireless communication protocols and interfaces.

The digitized signals received by the navigation interface 240 represent magnetic field information from the at least one electromagnetic field generator 212 detected by the at least one electromagnetic sensor 216. In the embodiment illustrated in FIG. 2, the navigation interface 240 transmits the digitized signals to a tracker module 250 over a local interface 215. The tracker module 250 calculates position and orientation information based on the received digitized signals. This position and orientation information provides a location of a surgical instrument or implant.

The tracker module 250 communicates the position and orientation information to a navigation module 260 over a local interface 215. As an example, this local interface 215 is a Peripheral Component Interconnect (PCI) bus. However, according to various alternate embodiments, equivalent bus technologies may be substituted.

Upon receiving the position and orientation information, the navigation module 260 is used to register the location of the surgical instrument or implant to acquired patient data. In the embodiment illustrated in FIG. 2, the acquired patient data is stored on a disk 245. The acquired patient data may include computed tomography data, magnetic resonance data, positron emission tomography data, ultrasound data, X-ray data, or any other suitable data, as well as any combinations thereof. By way of example only, the disk 245 is a hard disk drive, but other suitable storage devices may be used.

The acquired patient data is loaded into memory 220 from the disk 245. The acquired patient data is retrieved from the disk 245 by a disk controller 265. The navigation module 260 reads from memory 220 the acquired patient data. The navigation module 260 registers the location of the surgical instrument or implant to acquired patient data, and generates image data suitable to visualize the patient image data and a representation of the surgical instrument or implant. The image data is transmitted to a display controller 230 over a local interface 215. The display controller 230 is used to output the image data to display 228.

In another embodiment, the imaging and navigation system 200 may include an imaging apparatus 270 coupled to an imaging interface 275 for receiving real-time imaging data. The imaging data is processed in an imaging module 280. The imaging apparatus 270 provides the ability to display real-time position and orientation information of a surgical instrument or implant on the display 228.

While one display 228 is illustrated in the embodiment in FIG. 2, alternate embodiments may include various display configurations. Various display configurations may be used to improve operating room ergonomics, display different views, or display information to personnel at various locations.

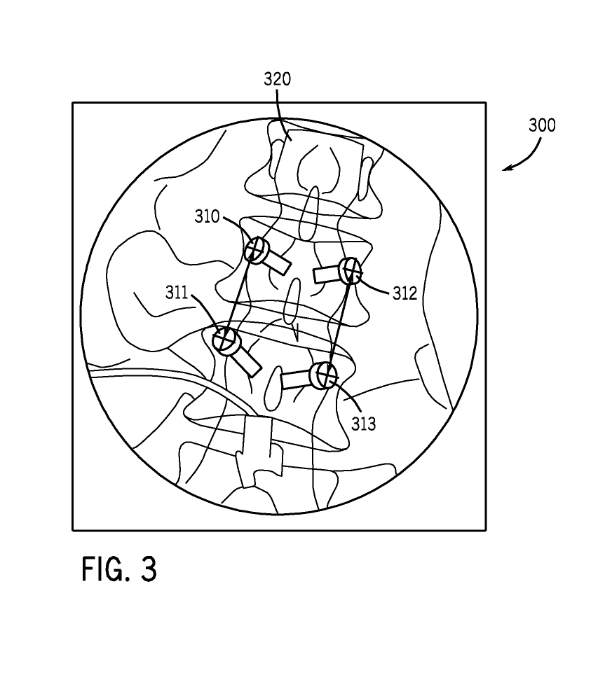

FIG. 3 is an exemplary display of an example of a user interface, such as a display, displaying an image with implant position and orientation information superimposed on the image.

In spinal surgery, pedicle screws are often utilized to stabilize the spine. Typically, these pedicle screws are driven through the pedicles and connected adjacently by interconnecting rods to manipulate and stabilize the spine during fusion between the bony segments of the spine.

These spinal procedures can incorporate surgical navigation technology wherein the location of a surgical instrument is measured and virtually superimposed on an image. The image may be pre-recorded, near real-time, or real-time, and is preferably obtained using known imaging technology such as X-ray, computed tomography (CT), magnetic resonance (MR), positron emission tomography (PET), ultrasound, or any combination thereof.

As illustrated in FIG. 3, a user interface, such as a display, shows a real-time (or substantially real-time due to an inherent system delay) position and orientation of a model or representation of the implant (e.g., a pedicle screw) on a 2D fluoroscopic image, for example. The position and orientation of the implant model may also be displayed on a registered 3D image dataset such as a CT scan. The implant model may appear as a line rendering, a few simply shaded geometric primitives (e.g., a parametric model containing two cylinders representing the screw head and body of a pedicle screw), or a realistic 3D model from a computer-aided design (CAD) file, for example.

Regardless of the visualization using to depict the implant, the implant model includes representations of key features of the implant that may be used for subsequent measurements. For example, the screw model includes a point feature for a center of a rod slot in the screw head. Additionally, the model may include a vector feature describing the orientation of the opening or slot extending through the screw heads for receiving an interconnecting rod therein.

As an example of a spinal fusion procedure, a surgeon uses a navigated surgical instrument to place a first screw into a vertebra. The surgeon then places a second screw into an adjacent vertebra. Each time a screw is placed, the screw's position and orientation is stored on the navigation system with respect to the local reference frame. This process can be repeated for all vertebral levels involved in the surgical procedure. The position includes X, Y and Z coordinates, while the orientation includes roll, pitch and yaw.

Once two or more screws are placed, measurements can be made between the positions and orientations of the screws, and features of the screws. For example, a simple calculation to determine the proper rod length of an interconnecting rod through the screws may be made using the cumulative distance between a series of screws (e.g., for a series of three screws (1, 2, 3), the cumulative distance would be |pt2−pt1|+|pt3−pt2|).

As another example, three or more screws have rod slot points that can be fit to a curve. Calculation of this curve can be used to select an appropriately shaped interconnecting rod. The orientation of the rod slot in two or more screws can also be determined to aid in the placement of the rod through the openings or slots in the screw heads.

Thus, certain embodiments enable quick, simultaneous, noninvasive measurements of multiple features of implants, thereby saving valuable time and risks of infection. Certain embodiments provide intraoperative measurements made from navigated placement of implants and instrumentation.

The method is used to measure and calculate positions and orientations for the placement of implants and instrumentation. In an embodiment, a software planning method aids a surgeon in minimally invasive percutaneous placement of pedicle screw insertions and placement of pedicle screw interconnecting rods. The pedicle screws are placed in adjacent vertebral levels and secured to one another by an interconnecting rod. The surgeon chooses the length and width of the implant based upon visual calculations of the pedicle and vertebral sizes calculated to the sagittal plane of each specific vertebral segment taking into consideration any rotational deformity.

The software planning method aids in determining the best functional placement of the pedicle screws and later for the placement of the pedicle screw interconnecting rods. The software calculates from multiplanar reconstructed CT images registered to the patient's anatomy by the morphometric dimensions of each vertebral segment to submillimeter accuracy. With an accurate representation of each of the vertebral segment dimensions, the surgeon can plot and mark on the X-ray anatomy the entry points, the vectors, the depth and height for the final positioning of each pedicle screw implant. In each vertebral segment, the goal is to place two pedicle screws percutaneously under X-ray navigation through a narrow bony channel (pedicle) from a posterior direction to an anterior direction into the vertebral body. These screws enter from both left and right sides of each vertebral segment at specific angles and the result must be an ideal or close to ideal convergence angle and depth for final positioning. This provides the greatest pullout strength for the implant screws in the bone and maximizes the success for fusion, construct strength and longevity of the implants. In specific indications such as spondylolithesis when one vertebral body segment shifts forward over another, a change in the linear height and depth between one vertebral level and another adjacent level provides a significant challenge to reduce the slippage back to the normal sagittal alignment plane between the levels. In this situation, a surgeon may use a special implant screw to manage the reduction and realignment. The software will not only assist the surgeon in this effort, but also confirm that the pedicle screw implant head is now level in the correct sagittal curve with the adjacent screw heads to ensure the passage of the rods on both sides of the spine will be accomplished successfully on the first attempt.

As shown, for example, in FIG. 3, a plurality of screws 310, 311, 312, 313 are placed in a plurality of vertebrae in a patient's spine 320. Position and orientation measurements of the implanted screws may be determined automatically by the imaging and navigation system and/or in conjunction with a user initiation or selection (e.g., by a user trigger based on a point and click, button click, pressure on a surgical instrument, keyboard selection, mouse selection, or other input selection, etc.).

In certain embodiments, position and orientation data may be determined for implants in real-time or substantially in real-time as the screws are placed by the user. For example, an implant center point, such as a center of an implant screw head, may be identified and used for proper placement of the implants.

Pedicle screw interconnecting rods may be inserted through openings or slots in the screw heads to facilitate spinal fusion. These rods are available in a variety of lengths, sizes and curvatures, and may be bent and/or cut to a variety of lengths, sizes and/or curvatures, for example. Based on position and orientation data from the placed screws, a user is provided with measurement data between the screws to aid in determining the proper rod length, size and/or curvature, as well as aid in placement targeting of the rod ends through the openings or slots in the individual screw head positions. The majority of these pedicle screw heads are polyaxial or adjustable to a fixed receiving angle, the computer will calculate these screw head angles to receive the rod ends for the surgeon's correct insertion path.

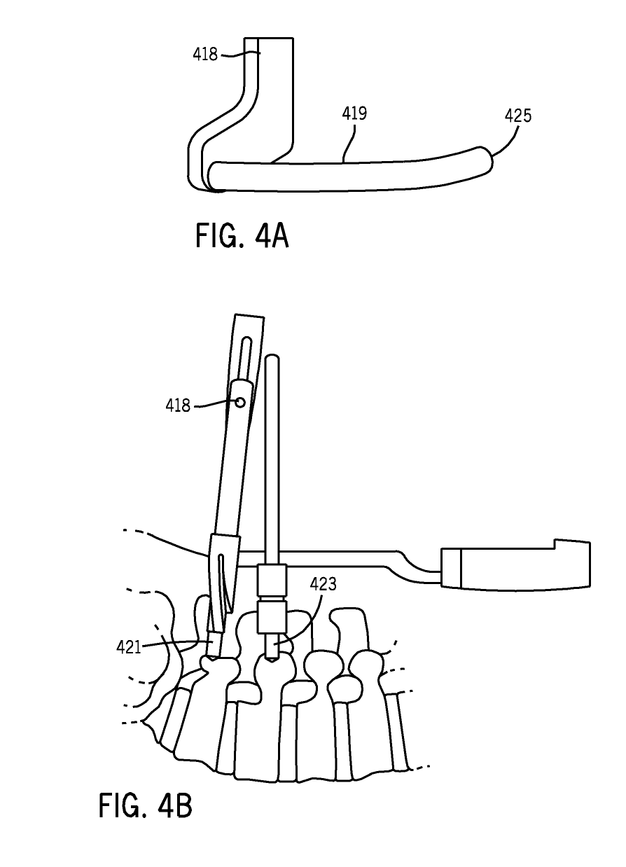

FIG. 4A is an exemplary diagram of an embodiment of a surgical instrument 418, a pedicle rod holder, holding an pedicle screw interconnecting rod 419. FIG. 4B is an exemplary diagram illustrating an embodiment of the placement of a pedicle rod 419 within at least two pedicle screw heads 421, 423. The navigation system tracks the vector tip 425 of the rod 419. Passage of the rod 419 through the skin and tissue, and into the aligned screw heads is accomplished through measurement of inter-pedicle distances and implant head distances, heights, planes, and rod size.

The method is used to measure and calculate positions and orientations for the placement of implants and instruments. In an embodiment, a software planning method aids a surgeon in passage of the rod through the tissue and into the aligned screw heads. This is accomplished by measurements of “inter-pedicle distances” and “implant head distances” (or in other words, the distance between the final implant screws in any segmental vertebrae up to any number necessary). Also included in the method is the height and planes of the screw heads, and rod sizes to fit the curvature of the given spine at any given level of the spine as well as the angle of the screw heads that may be of a style that is a fixed solid screw implant head type or a multi-axial adjustable screw head type that needs to be considered for any planning to work accurately. The key is to line up the screw head angles using the software and X-ray calculations pre-op and intra-op for a straight path for the rod to glide through the screw heads not only by their angles or in-between distances, but also by their sagittal plane height of each.

The software planning method also aids in determining the best functional placement of the rods into each implant screw head and between each segmental level on both sides of the dorsal spine. The surgeon chooses the length and width of the implant based upon visual calculations of the pedicle and vertebral sizes. The goal is to pass the rods through the skin and between the muscles into and through each pedicle implant screw head on the first try with little to no guessing that it will land in each pedicle securely in the least amount of time. Based on implant position and orientation information and distance measurements between implants, a surgeon may determine an appropriate rod length and/or curvature for placement between implant positions.

In certain embodiments, a user may be provided with suggested types, lengths, angles, and/or curvatures of rod or other connector joining two or more implants, such as screws and or guide sleeves. In certain embodiments, a user may also be guided in placement of such connector.

Making approximate measurements for anatomical differences and rod length with or without jigs (rod guide sleeves) are not fully reliable and direct access to the implanted screws can be problematic in percutaneous MIS procedures and ultimately for desired compression and/or distraction and are therefore also prone to trial-and-error methods. Tracking and calculating the best angles of guide sleeves connected to implant screws in specific MIS fusion procedures may benefit the procedure by quicker placement and better seating of the implant rods to the implant screws maximizing the goal for a proper compression and/or distraction of the vertebral segment/s. Future variations of this navigation may obviate the need for guide sleeves simplifying and minimizing the necessary procedural components and steps.

Rather than directly and explicitly measuring anatomical distances, implant hardware is measured and a relationship between the implant and the anatomy is utilized. For example, screws may be inserted in bone at different heights, so a curved rod is to be inserted to connect the screws. Measurements based on screw position and orientation may identify the distance and curvature to be used and provide such data to the user for rod selection.

In certain embodiments, a measurement may be identified through positioning of a navigated or otherwise tracked tool with respect to the image of the patient anatomy, touch screen selection with respect to the image, keyboard selection and/or mouse selection, for example. In an embodiment, a user positions a navigated or tracked surgical instrument with respect to the image of the patient anatomy, such as the image of FIG. 3. When the surgical instrument is aligned or substantially aligned with a measurement, that measurement is determined for the user.

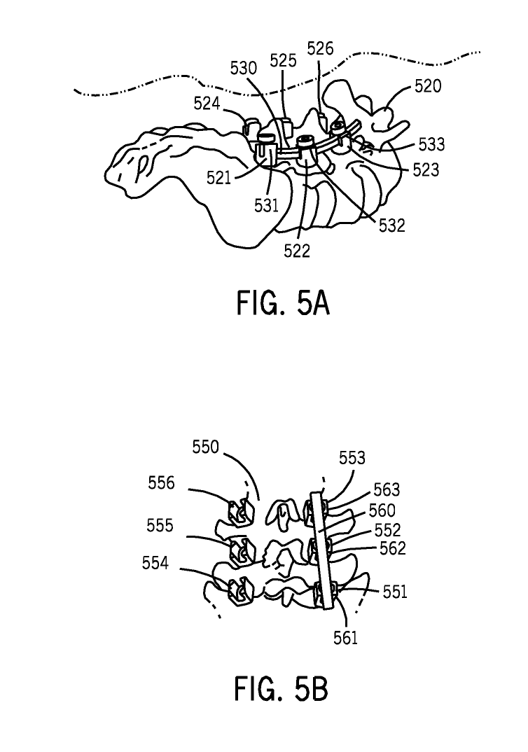

FIG. 5A is an exemplary diagram illustrating an embodiment of a portion of a spine 520 with pedicle screws 521, 522, 523, 524, 525, 526 and a pedicle rod 530 placed within pedicle screw heads 531, 532, 533. FIG. 5B is an exemplary diagram illustrating an embodiment of a portion of a spine 550 with pedicle screws 551, 552, 553, 554, 555, 556 and a pedicle rod 560 placed within pedicle screw heads 561, 562, 563. As described above, a curved rod 530, as illustrated in FIG. 5A, may be placed by a user between screw heads 531, 532, 533 (e.g., between center points of the screw heads). Alternatively, a straight rod 560, as illustrated in FIG. 5B, may be placed by a user between screw heads 561, 562, 563 (e.g., between center points of the screw heads). FIGS. 5A and 5B illustrate different types and sizes of pedicle screws and pedicle rods. The position and orientation of the screws may be known due to navigation/tracking information, as described above, and/or through image processing without navigation, for example.

In certain embodiments, position and orientation of an implant may be measured and/or represented in 2D space, 3D space and/or a combination of 2D and 3D space, for example. In certain embodiments, position and distance measurement data may be presented to a user in an absence and/or aside from an image display.

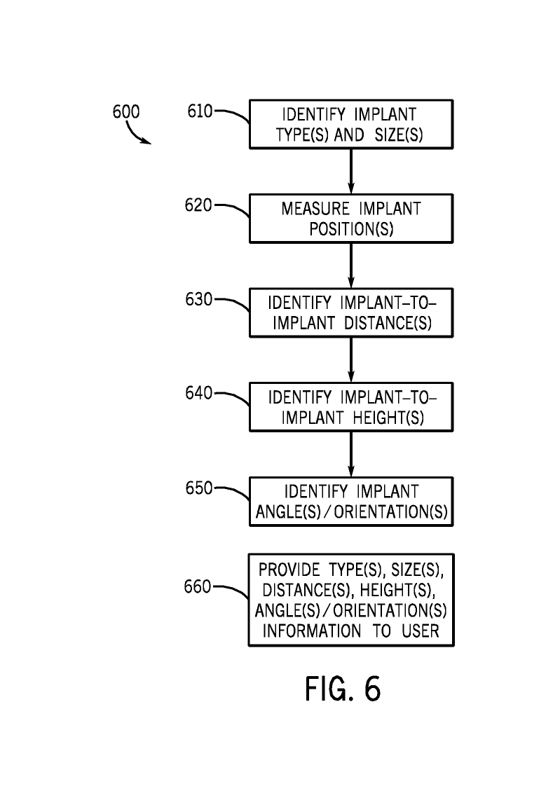

FIG. 6 is an exemplary flowchart of an embodiment of a method 600 for measuring and calculating implant position and orientation information. At step 610, implant types and sizes are identified. At step 620, implant positions are measured. For example, position and orientation information for a plurality of pedicle screws implanted in a patient's spine may be measured. Implant representations may be superimposed on an image of a patient's anatomy for user review pre-operatively and intraoperatively. At step 630, an implant-to-implant distance measurement is identified and calculated. The distances between each implant are measured and calculated. At step 640, an implant-to-implant height measurement is identified and calculated. This may include individual height measurements for each individual implant, or a comparison of height measurements for two or more implants. In addition to the height measurements and calculations, the sagittal plane height of each implant is also measured and calculated. At step 650, an implant-to-implant angle and/or orientation measurements are identified and calculated. This may include individual angle and/or orientation measurements for each individual implant, or a comparison of angle and/or orientation measurements for two or more implants and or instruments such as guide sleeves.

At step 660, type, size, distance, height, angle and/or orientation measurement information is provided to a user. For example, type, size, distance, height, angle and/or orientation measurement information between one or more pedicle screws may be displayed on an image and/or provided in addition to an image for surgeon review pre-operatively and intraoperatively in determining an appropriate interconnecting rod length and/or curvature. As another example, alternatively and/or in addition, distance measurement information may be provided as one or more recommendations regarding rod selection, such as suggested rod length and/or curvature. Navigation may be employed to provide measurement information instead and/or in addition.

For example, a calculation of rod length may be determined using a cumulative distance between a series of screws (e.g., for three screws 1, 2, 3 the cumulative distance would be |pt2−pt1|+|pt3−pt2|). Additionally, three or more screws have rod slot points that can be fit to a curve. Calculation of curvature may be used to select and/or suggest an appropriately shaped rod. An orientation of the rod slot in two or more screws may also be used for determination of curvature, for example.

Additionally, pedicle screw and/or other implant placement may be stored to aid in subsequent implant placement. For example, a placement location of a pedicle screw may be stored or otherwise maintained while placing additional screws at adjacent levels. Knowing prior placement at adjacent levels may help subsequent screws to be driven to like depths and angles. Thus, insertion of an interconnecting rod between the screws may be improved.

Thus, certain embodiments provide workflow enhancement for surgical navigation and measurement. For example, the distance between two pedicle screw heads is used to determine the size of the interconnecting rod. Navigation helps improve workflow to measure the distance rather than manual measurement via calipers and a sizing template. Additionally, navigated pedicle screws may be graphically rendered and represented as an overlay on an image for viewing by a clinician. The overlay helps maintain visualization of screw and/or other implant locations, for example.

Certain embodiments may operate in conjunction with a 2D/3D hybrid navigation system incorporates real-time updating and ease of use of a 2D system along with an easily registered 3D CT or 3D fluoroscopic datasets. Safety and precision of medical procedures may be enhanced with a 2D/3D navigation system. Use of a CT dataset along with 2D intraoperative imaging adds to visualization and understanding of an anatomy in an operating room. Such a system may have applicability in a variety of medical procedures, such as spinal procedures, cranial procedures, orthopedic procedures, and other clinical procedures. Spinal procedures may include posterolateral open and minimally invasive surgical (MIS) pedicle screws, posterior C1-C2 transarticular screw fixation, transoral odontoid fixation, cervical lateral mass plate screw fixation, anterior thoracic screw fixation, scoliosis, kyphosis, kyphoplasty, vertebroplasty, transforaminal lumbar interbody fusion (TLIF), artificial disks, burst fractures, excision of paraspinal neoplasms, etc.

Although the systems and methods described herein may be used with a variety of implants, an example of a screw (and more specifically a pedicle screw) is used for convenient purposes of illustration only. Such an example is not intended to limit the embodiments disclosed and encompassed herein to screw implants. For example, systems and methods may be used in conjunction with insertion of a stent into a patient blood vessel. A wire or other guide may be fed into the vessel with markings on the wire to allow navigated measurement of points along the wire. Distance measurement along the wire may be sued to recommend and/or aid in determination of stent and/or balloon size, for example. In certain embodiments, any hardware introduced into a patient for which position measurements may be obtained may be used in conjunction with distance measurement as described above.

The embodiments described herein provide a surgeon with the confidence for intra-operative planning with the assistance of computer navigation for patient specific anatomical dimensions in the placement of percutaneous MIS instrumentation. The invention further benefits the surgeon in the placement planning and confirmation of spinal implant instruments in the best alignments on the first attempt to minimize the use of X-ray dose and procedural time. The end benefit would be the ideal placement and alignments between these implants for better strength and longevity of the implants leading to improved patient range of motion and better outcomes. The surgeon can in advance or on the fly plan the ideal trajectories for screw implant placements and use these measurements to further plan the next key factor steps in the placement of rods to be secured to the already implanted screws with out the postulated need for significant use times for X-ray fluoroscopy and matching the insertion angles of the rods to screws without the direct benefit of open visualization. The guess and feel factor even with X-ray use is removed and precision placements of both screws into bone and rods to these screws are better facilitated in theoretically less overall X-ray and operative times.

Several embodiments are described above with reference to drawings. These drawings illustrate certain details of specific embodiments that implement the systems and methods and programs of the present invention. However, describing the invention with drawings should not be construed as imposing on the invention any limitations associated with features shown in the drawings. The present invention contemplates methods, systems and program products on any machine-readable media for accomplishing its operations. As noted above, the embodiments of the present invention may be implemented using an existing computer processor, or by a special purpose computer processor incorporated for this or another purpose or by a hardwired system.

As noted above, embodiments within the scope of the present invention include program products comprising machine-readable media for carrying or having machine-executable instructions or data structures stored thereon. Such machine-readable media can be any available media that can be accessed by a general purpose or special purpose computer or other machine with a processor. By way of example, such machine-readable media may comprise RAM, ROM, PROM, EPROM, EEPROM, Flash, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such a connection is properly termed a machine-readable medium. Combinations of the above are also included within the scope of machine-readable media. Machine-executable instructions comprise, for example, instructions and data which cause a general purpose computer, special purpose computer, or special purpose processing machines to perform a certain function or group of functions.

Embodiments of the invention are described in the general context of method steps which may be implemented in one embodiment by a program product including machine-executable instructions, such as program code, for example in the form of program modules executed by machines in networked environments. Generally, program modules include routines, programs, objects, components, data structures, etc. that perform particular tasks or implement particular abstract data types. Machine-executable instructions, associated data structures, and program modules represent examples of program code for executing steps of the methods disclosed herein. The particular sequence of such executable instructions or associated data structures represents examples of corresponding acts for implementing the functions described in such steps.

Embodiments of the present invention may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communication protocols. Those skilled in the art will appreciate that such network computing environments will typically encompass many types of computer system configurations, including personal computers, hand-held devices, multi-processor systems, microprocessor-based or programmable consumer electronics, network PCs, minicomputers, mainframe computers, and the like. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

An exemplary system for implementing the overall system or portions of the invention might include a general purpose computing device in the form of a computer, including a processing unit, a system memory, and a system bus that couples various system components including the system memory to the processing unit. The system memory may include read only memory (ROM) and random access memory (RAM). The computer may also include a magnetic hard disk drive for reading from and writing to a magnetic hard disk, a magnetic disk drive for reading from or writing to a removable magnetic disk, and an optical disk drive for reading from or writing to a removable optical disk such as a CD ROM or other optical media. The drives and their associated machine-readable media provide nonvolatile storage of machine-executable instructions, data structures, program modules and other data for the computer.

The foregoing description of embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principals of the invention and its practical application to enable one skilled in the art to utilize the invention in various embodiments and with various modifications as are suited to the particular use contemplated.

Those skilled in the art will appreciate that the embodiments disclosed herein may be applied to the formation of any medical navigation system. Certain features of the embodiments of the claimed subject matter have been illustrated as described herein, however, many modifications, substitutions, changes and equivalents will now occur to those skilled in the art. Additionally, while several functional blocks and relations between them have been described in detail, it is contemplated by those of skill in the art that several of the operations may be performed without the use of the others, or additional functions or relationships between functions may be established and still be in accordance with the claimed subject matter. It is, therefore, to be understood that the appended claims are intended to cover all such modifications and changes as fall within the true spirit of the embodiments of the claimed subject matter.

While the invention has been described with reference to several embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

What is claimed is:

1. A method for placement of an implant, said method comprising:
measuring a position of at least two implants;
calculating a distance between the position of the at least two implants; and
displaying the distance to a user.

2. The method of claim 1, further comprising measuring a height of the at least two implants.

3. The method of claim 1, further comprising measuring a sagittal plane of the at least two implants.

4. The method of claim 1, further comprising measuring an angle of the at least two implants.

5. The method of claim 1, further comprising measuring an orientation of the at least two implants.

6. The method of claim 1, further comprising fitting said positions of the at least two implants to a curve.

7. The method of claim 1, further comprising measuring the positional distance and angles of each instrument guide sleeve to the pedicle screw heads.

8. The method of claim 1, further comprising suggesting a connecting rod length based on the distance.

9. The method of claim 6, further comprising suggesting a curvature of a connecting rod based on the curve.

10. The method of claim 1, further comprising highlighting the distance on an image display.

11. The method of claim 1, further comprising storing the position and distance information for subsequent implant placement.

12. The method of claim 1, wherein the at least two implants are graphically rendered and overlaid on an image with trajectory information and distance information.

13. A system for placement of at least one implant, said system comprising:
an imaging system configured for taking at least one image of a patient;
a navigation system configured for tracking position and orientation of at least one implant;
a computer configured to measure and calculate the position and orientation of the at least one implant; and
a display configured to display the at least one image of the patient and superimpose a graphical representation of the at least one implant with position and orientation information of the at least one implant on the at least one image of the patient.

14. The system of claim 13, wherein the computer recommends an interconnection component characteristic based on the position and orientation information.

15. The system of claim 14, wherein said interconnection component characteristic comprises at least one of component length and component curvature.

16. The system of claim 13, wherein the computer stores position and orientation information of the at least one implant for subsequent implant placement.

17. The system of claim 13, wherein the display includes trajectory and distance information for subsequent implant placement.

18. The system of claim 13, wherein the computer receives tracking information for the at least one implant.

19. A computer-readable medium having a set of instructions for execution on a computer, said set of instructions comprising:
a tracking routine for measuring the position and orientation of at least one implant;
a measurement routine for measuring differences between the position and orientation of the at least one implant;
a calculation routine for calculating curves and trajectories for implant placement; and
a display routine for displaying an image of a patient and superimposing on the image a graphical representation of the at least one implant with position and orientation information, and curve and trajectory information.

20. A method for implant placement and measurement, said method comprising:
measuring the positions (x, y, z) of at least two implants; and
calculating a distance between the two positions of the at least two implants.

21. A method for implant placement and measurement, said method comprising:
measuring the positions (x, y, z) of three or more implants; and
calculating a best fit curve between the three or more positions of the three or more implants.

22. A method for implant placement and measurement, said method comprising:
measuring the positions (x, y, z) of at least two implants;
measuring the orientations (roll, pitch yaw) of the at least two implants; and
calculating a best fit curve between the at least two positions of the at least two implants and the at least two orientations of the at least two implants.
